(19) United States
(12) Patent Application Publication
(10) Pub. No.: US 2021/0236373 A1
(43) Pub. Date: Aug. 5, 2021

(54) HYBRID SPRING AND MASS COUNTERBALANCING ORTHOTIC

(71) Applicant: Abilitech Medical, Inc., Eden Prairie, MN (US)

(72) Inventors: Mark Oreschnick, Inver Grove Heights, MN (US); Eli Krumholz, Minneapolis, MN (US); Julie Lundstrom, Ellsworth, WI (US); Shawna Persaud, Lakeville, MN (US); Angie Conley, Minneapolis, MN (US)

(21) Appl. No.: 17/161,829

(22) Filed: Jan. 29, 2021

Related U.S. Application Data
(60) Provisional application No. 62/967,927, filed on Jan. 30, 2020.

(51) Int. Cl. A61H 1/02; B25J 9/00

(52) U.S. Cl. CPC A61H 1/0281; F16H 25/20; B25J 9/0006

(57) ABSTRACT

An upper torso augmentation device in which a moment of an arm assembly is tunable by the movement of one or more movable masses. The upper torso augmentation device including an upper arm assembly pivotably coupled to a shoulder assembly, the upper arm assembly including an assisted force mechanism configured to aid in counteracting an effect of gravity upon the upper arm assembly and any payload carried thereby, the assisted force mechanism comprises one or more movable masses configured to move relative to a distal end of the upper arm assembly, thereby affecting a change in a moment of the upper arm assembly.

### RELATED APPLICATION INFORMATION

This application claims the benefit of U.S. Provisional Application No. 62/967,927, filed Jan. 30, 2020, the contents of which are fully incorporated herein by reference.

### TECHNICAL FIELD

The present disclosure relates generally to systems and methods for upper extremity lift and assist of patients suffering from a loss of motor skills. More particularly, the present disclosure relates to an upper torso augmentation system and method of use, configured to augment existing upper body movement and rebuild lost motor skills in patients suffering from neuromuscular disorders, spinal injuries, or impairment of limbs as a result of a stroke.

### BACKGROUND

Individuals with neuromuscular abnormalities, such as neuromuscular disorders, spinal injuries, or impairment of limbs as a result of a stroke, often experience muscular atrophy and/or impaired motor function, which can lead to a loss of full functionality in their limbs and upper body. Such a loss in functionality can make the performance of routine tasks difficult, thereby adversely affecting the individual's quality of life.

In the United States alone, 1.4 million people suffer from neuromuscular disorders. It is estimated that approximately 45,000 of these people are children, who are affected by one or more pediatric neuromuscular disorders. Pediatric neuromuscular disorders include Spinal Muscular Atrophy (SMA), cerebral palsy, Arthrogryposis Multiplex Congenital (AMC), Becker Muscular Dystrophy, and Duchenne Muscular Dystrophy (DMD). Adult neuromuscular diseases include Multiple Sclerosis (MS), Amyotrophic Lateral Sclerosis (ALS) and Facioscapulohumeral Muscular Dystrophy (FSHD). Many of these muscular disorders are progressive, such that there is a slow degeneration of the spinal cord and/or brainstem motor neurons resulting in generalized weakness, atrophy of skeletal muscles, and/or hypotonia.

In the United States, approximately 285,000 people suffer from spinal cord injuries, with 17,000 new cases added each year. Approximately 54% of spinal cord injuries are cervical injuries, resulting in upper extremity neuromuscular motor impairment. Spinal cord injuries can cause morbid chronic conditions, such as lack of voluntary movement, problematic spasticity, and other physical impairments which can result in a lower quality of life and lack of independence.

In the United States, it is estimated that there are over 650,000 new surviving stroke victims each year. Approximately 70-80% of stroke victims have upper limb impairment and/or hemiparesis. Numerous other individuals fall victim to Silent Cerebral Infarctions (SCI), or “silent strokes,” which can also lead to progressive limb impairment. Complications from limb impairment and hemiparesis may involve spasticity, or the involuntary contraction of muscles when individuals try to move their limbs. If left untreated, the spasticity can result in the muscles freezing in abnormal and painful positions. Also, following a stroke, there is an increased possibility of developing hypertonicity, or the increased tightness of muscle tone.

People afflicted with neuromuscular abnormalities often exhibit diminished fine and gross motor skills. In cases where a person is capable of only asymmetric control of the particular joint, the person may be able to control the muscle group responsible for flexion about the joint, but his or her control over the muscle group responsible for extension may be impaired. Similarly, the opposite may be true, in that the user may have control in the extension direction, but not in the flexion direction. In either case, if the person cannot exert his or her triceps or release a hyperactive bicep, the person may be unlikely to perform the task they desire. Even in cases where a person retains symmetric control over a joint, the person may be left with reduced control over muscle groups on opposite sides of the joint. As a result, the person may be incapable of achieving the full range of motion that the joint would normally permit and/or be incapable of controlling the joint so that the associated limb segments exert the amount of force required to perform the desired task.

In many cases, a reduction in strength or impairment of motor function, as a result of neuromuscular abnormalities, can be slowed, stopped, or even reversed through active treatment and therapy. At least for stroke victims, data suggests that the sooner that the therapy is started after the impaired motor function is first noticed, and the greater the amount of therapy that is performed by the patient, the more likely the patient is to have a better recovery. Unfortunately, the therapy often utilizes expensive equipment and is limited to in-clinic settings, thereby significantly restricting the amount of therapy that can be performed by the patient. In other cases, such as with progressive neuromuscular disorders, the goal of the treatment may be to slow the decline in functionality, so as to maintain the individual's quality of life for as long as possible. Common treatment methods include physical therapy combined with medications to provide symptomatic relief.

Regarding spinal cord injuries, while there are no known treatments that can reverse morbidities, repetitive high-intensity exercise and the use of orthoses have been used to improve the strength and overall neuromuscular health of patients. In particular, a number of upper arm support devices have been developed to strengthen upper extremities and improve independence for accomplishing activities of daily living. Examples of such orthoses are disclosed in Published PCT Application Nos. WO2018111853; WO2018165413; WO2020086515 (assigned to the Applicant of the present disclosure), the contents of which are hereby incorporated by reference herein.

Although such advanced orthotic systems have proven to work well, there remains a need for improvements in body frames configured to be worn around a torso and/or upper extremity of the user to provide support for the orthotic device. The present disclosure addresses this concern.

### SUMMARY OF THE DISCLOSURE

Embodiments of the present disclosure provide an upper torso augmentation device configured to counterbalance the weight of an arm of the user and aid movement of the arm. The upper torso augmentation device can include one or more movable counterbalancing weights or masses configured to affect a moment arm change to counteract one or more spring constants under a given load.

One embodiment of the present disclosure provides an upper torso augmentation device in which a moment of an arm assembly is tunable by the movement of one or more movable masses. The upper torso augmentation device can include an upper arm assembly pivotably coupled to a shoulder assembly, the upper arm assembly including an assisted force mechanism configured to aid in counteracting an effect of gravity upon the upper arm assembly and any payload carried thereby, wherein the assisted force mechanism comprises one or more movable masses configured to move relative to a distal end of the upper arm assembly, thereby affecting a change in a moment of the upper arm assembly.

In one embodiment, the assisted force mechanism comprises at least one spring. In one embodiment, a tension in the at least one spring is adjustable via a pre-tensioning mechanism. In one embodiment, the upper torso augmentation system further includes a lower arm assembly pivotably coupled to the upper arm assembly, the lower arm assembly including a second assisted force mechanism configured to aid in counteracting an effect of gravity upon the lower arm assembly and any payload carried thereby, wherein the second assisted force mechanism comprises one or more lower arm movable masses configured to move relative to a distal end of the lower arm assembly, thereby affecting a change in moment of the lower arm assembly.

In one embodiment, the one or more movable masses are moved via at least one of a manual or automated actuation system. In one embodiment, the assisted force mechanism is controllable via a user interface. In one embodiment, the assisted force mechanism further comprises one or more sensor configured to identify known payloads for automatic movement of the one or more movable masses. In one embodiment, the assisted force mechanism includes one or more load cells configured to monitor forces experienced in an arm of a user, wherein a deviation from an expected force value triggers automatic movement of the one or more movable masses. In one embodiment, the assisted force mechanism is configured to provide active resistance as a form of resistance training. In one embodiment, the assisted force mechanism is configured to calculate an amount of work performed by a user over a defined period of time.

One embodiment of the present disclosure provides an upper torso augmentation device, including at least one arm assembly including an assisted force mechanism configured to counteract an effect of gravity upon an arm of a user through a desired range of motion, the assisted force mechanism comprising one or more movable masses configured to move relative to a distal end of the at least one arm assembly, thereby affecting a change in moment of the at least one arm assembly.

In one embodiment, the assisted force mechanism includes an actuation system comprising a rotatable lead screw to shift the one or more movable masses along a track. In one embodiment, the assisted force mechanism includes an actuation system comprising a pulley wheel system configured to drive a cable upon which the one or more movable masses is attached in order to affect movement in the one or more movable masses along a length of the at least one arm assembly. In one embodiment, the assisted force mechanism includes an actuation system comprising a rack and pinion system configured to affect movement in the one or more movable masses along a length of the at least one arm assembly. In one embodiment, the assisted force mechanism includes an actuation system comprising a resilient push pull linkage configured to affect movement in the one or more movable masses along the length of the at least one arm assembly.

The summary above is not intended to describe each illustrated embodiment or every implementation of the present disclosure. The figures and the detailed description that follow more particularly exemplify these embodiments.

### BRIEF DESCRIPTION OF THE DRAWINGS

The disclosure can be more completely understood in consideration of the following detailed description of various embodiments of the disclosure, in connection with the accompanying drawings, in which:

FIG. 1 is a profile view depicting an upper torso augmentation device including an assisted force mechanism configured to adjust a limb augmentation counterbalancing force, in accordance with an embodiment of the disclosure.

FIG. 2 is a partial schematic diagram depicting an upper torso augmentation device, in accordance with an embodiment of the disclosure.

FIG. 3 is a partial, schematic diagram depicting an upper torso augmentation device including one or more movable counterbalancing masses, in accordance with an embodiment of the disclosure.

FIG. 4 is a schematic, profile diagram depicting an upper torso augmentation device having one or more movable masses to tune an upper and lower moment of a corresponding upper and lower arm assembly, in accordance with an embodiment of the disclosure.

FIG. 5 is a schematic diagram depicting a sliding mass actuation system including a rotatable lead screw configured to longitudinally shifting movable mass along a portion of an upper torso augmentation device, in accordance with an embodiment of the disclosure.

FIG. 6 is a schematic diagram depicting a sliding mass actuation system including a pulley and track system configured to longitudinally shifting movable mass along a portion of an upper torso augmentation device, in accordance with an embodiment of the disclosure.

FIG. 7A is a schematic diagram depicting a sliding mass actuation system including a rack and pinion system configured to longitudinally shifting movable mass along a portion of an upper torso augmentation device, in accordance with an embodiment of the disclosure.

FIG. 7B is a close-up, schematic view of the rack and pinion system, in accordance with an embodiment of the disclosure.

FIG. 7C is a close-up, schematic view of an alternative rack and worm gear system, in accordance with an embodiment of the disclosure.

FIG. 8 is a schematic diagram depicting a sliding mass actuation system including a resilient push-pull linkage configured to longitudinally shifting movable mass along a portion of an upper torso augmentation device, in accordance with an embodiment of the disclosure.

FIG. 9 is a schematic diagram depicting a continuous mass transfer actuation system configured to transfer fluid as a movable mass along a portion of an upper torso augmentation device, in accordance with an embodiment of the disclosure.

FIG. 10 is a schematic diagram depicting a continuous mass transfer actuation system configured to transfer a continuous chain of solid media having multiple densities as a movable mass along a portion of an upper torso augmentation device, in accordance with an embodiment of the disclosure.

FIG. 11 is a schematic diagram depicting a continuous mass transfer actuation system configured to transfer a continuous chain of solid media having multiple densities into a coil at a distal end of an upper torso augmentation device, in accordance with an embodiment of the disclosure.

FIG. 12 is a schematic diagram depicting a continuous mass transfer actuation system including a pulley and track system configured to transfer a movable mass along a portion of an upper torso augmentation device, in accordance with an embodiment of the disclosure.

While embodiments of the disclosure are amenable to various modifications and alternative forms, specifics thereof shown by way of example in the drawings will be described in detail. It should be understood, however, that the intention is not to limit the disclosure to the particular embodiments described. On the contrary, the intention is to cover all modifications, equivalents, and alternatives falling within the spirit and scope of the subject matter as defined by the claims.

### DETAILED DESCRIPTION

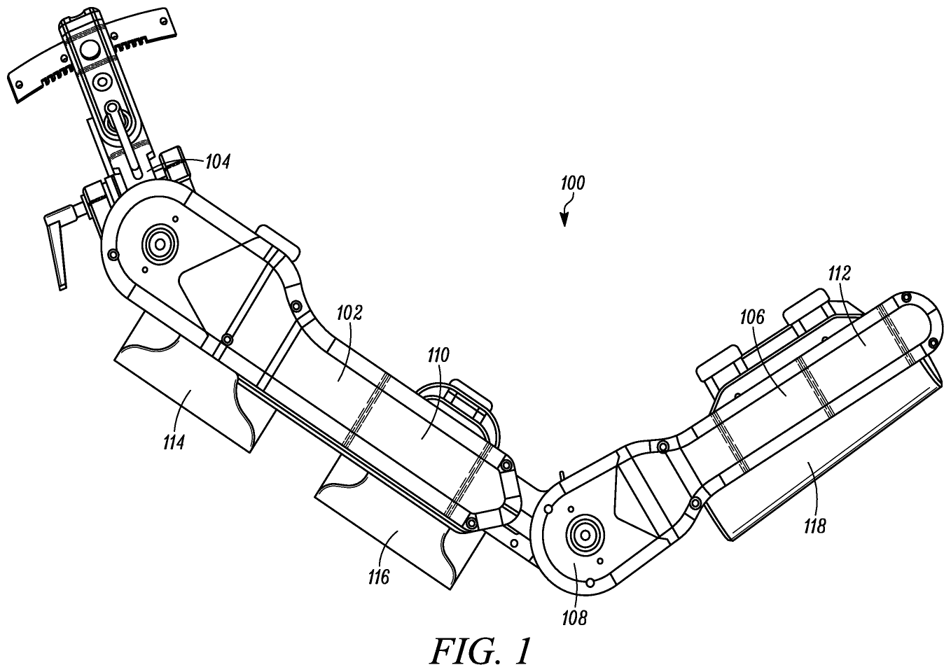

Referring to FIG. 1, an upper torso augmentation device 100 having one or more springs and movable weights configured to adjust a limb augmentation counterbalancing force, is depicted in accordance with an embodiment of the disclosure. In one embodiment, the upper torso augmentation device 100 can include an upper arm assembly 102 pivotably coupled to a shoulder assembly 104. An optional lower arm assembly 106 can be pivotably coupled to the upper arm assembly 102 via an elbow assembly 108. In some embodiments, at least one of the upper arm assembly 102 and/or lower arm assembly 106 can include an assisted force mechanism 110, 112 (which can include one or more springs and/or movable weights as described in further detail below), wherein an output of the assisted force mechanism 110, 112 is adjustable, thereby enabling an output of the assisted force mechanism 110, 112 to approximate a determined minimum assist force required for the patient to move their arm through a desired range of motion so as to minimize any excess torque produced by the upper torso augmentation device 100 necessary to overcome the effects of gravity.

As further depicted in FIG. 1, the upper torso augmentation device 100 can also include one or more cuffs 114, 116 & 118 configured to support portions of a user's arm in connection to the upper torso augmentation device 100, as well as to transfer motion of the upper torso augmentation device 100 into the human body. In one embodiment, the one or more cuffs can include a humeral cuff 114, elbow cuff 116, and a forearm cuff 118. For improved adaptability and conformability of the upper torso augmentation device 100 to a wide variety of patient shapes and sizes, a variety of cuff sizes and shapes can be provided. Additionally, embodiments of the present disclosure can enable adjustment in the positioning of the cuffs 114, 116 & 118 for improved fitting of the upper torso augmentation device 100 to a body of a patient.

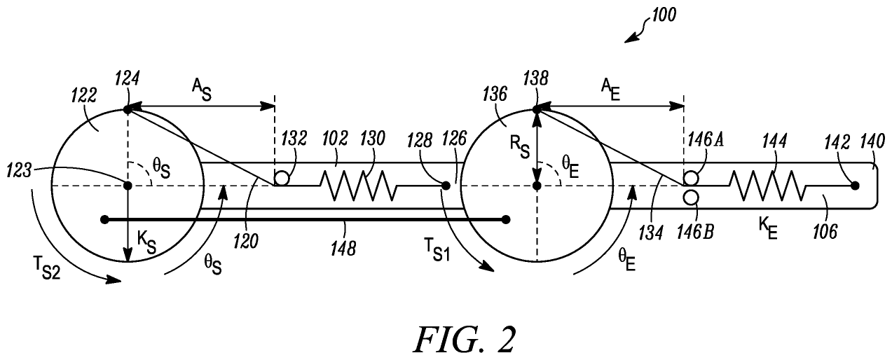

Referring to FIG. 2, a partial schematic diagram of an upper torso augmentation device 100 is depicted in accordance with an embodiment of the disclosure. In one embodiment, the upper arm assembly 102 can include a tension cable 120 anchored to an indexing disk 122 at a first end 124 and to a distal end 126 of the upper arm assembly 102 at a second end 128 via a spring 130. In some embodiments, the tension cable 120 can travel around one or more bearings 132 or pulleys between the first end 124 and the second end 128.

Similarly, the optional lower arm assembly 106 can include a tension cable 134 anchored to an indexing disk 136 at a first end 138 and to a distal end 140 of the lower arm assembly 106 at a second end 142 via a spring 144. In some embodiments, the tension cable 134 can travel around one or more bearings 146A/B or pulleys between the first end 138 and the second end 142. For example, in one embodiment, a pair of bearings 146A/B can be utilized to enable rotation of the lower arm assembly 106 beyond an angle at which the tension cable 134 would no longer be constrained by a single bearing 146A.

In some embodiments, a connecting rod 148 operably coupling the upper arm indexing disk 122 to the lower arm indexing disk 136 can be configured to rotate the lower arm indexing disk 136 based on the position of the upper arm indexing disk 122, thereby increasing or decreasing a tension in the lower arm tension cable 134 based on a shoulder rotation position (e.g., a lateral position with respect to a gravitational reference) of the upper arm assembly 102. For example, in some embodiments, the first indexing disk 122 can be configured to maintain its position with respect to a gravitational frame of reference, regardless of the shoulder rotation of the user and subsequent position of the upper arm assembly. Operably coupling the first indexing disk 122 to the second indexing disk 136 via the connecting rod 148, thus forces the second indexing disk 136 to also maintain its position with respect to a gravitational frame of reference. Accordingly, in some embodiments, the connecting rod 148 is configured to ensure that a counterbalance force of the lower arm assembly 106 (e.g., a tension preload in the lower arm spring 144) is adjusted based on a shoulder angle of the user.

When the first and second springs 130, 140 are properly preloaded (e.g., via rotation of the upper and lower indexing discs 122, 136) an “ideal” counterbalancing force can be achieved (e.g., a gravitational force exerted upon a user's arm can be completely counterbalanced, thereby creating the effects of weightlessness of the arm to the user), with alignment and friction between components of the upper torso augmentation device 100 being the primary elements negatively affecting an ideal counterbalance throughout an entire desired range of motion. In such embodiments, the spring counterbalance for the patient can be determined by computing a mechanical moment produced by a combination of the patient's arm and the upper torso augmentation device 100. The mechanical moment (also referred to herein as the “torque”) is defined as the total mass (of both the patient's arm and the device 100) multiplied by the distance from the pivot point 123, 137 (e.g., the center of the indexing discs 122, 136) to the center of gravity (CoG) of the total mass.

In some embodiments, the moment of the lower arm assembly 106 can be defined by the following formula:

Mo_{lower arm}=Sin Θ_{E}×((M_{lower arm}×CoG_{lower arm})+(M_{user arm}×CoG_{user arm})

Where, Mo_{lower arm} represents the moment of the lower arm assembly, Θ_{E} represents the flexion angle of the lower arm assembly 106, M_{lower arm} represents the mass of the lower arm assembly 106, CoG_{lower arm} represents the center of gravity of the mass of the lower arm assembly 106, M_{user arm} represents the mass of the user's lower arm, and CoG_{user arm} represents the center of gravity of the mass of the user's lower arm. The mass of the user's lower arm (and CoG) can include the patient's hand, as well as any payload in the hand.

In some embodiments, the moment of the upper arm assembly 102 can be defined by the following formula:

Mo_{upper arm}=MO_{lower arm}+(Sin Θₛ×((M_{upper arm}×COG upper arm)+(M_{upper arm}×CoG_{upper arm}))

Where, Mo_{upper arm} represents the moment of the upper arm assembly, ηₛ represents the flexion angle of the upper arm assembly 102, M_{upper arm} represents the mass of the upper arm assembly 102, CoG_{upper arm} represents the center of gravity of the mass of the upper arm assembly 102, M user arm represents the mass of the user's upper arm, and CoG user arm represents the center of gravity of the mass of the user's upper arm. Note that the above formulas may not account for abduction and adduction angles. Further, the formulas can be defined using sin( ) or cos( ) functions depending on the coordinate system used.

Adjusting the assisted force mechanism 110, 112 to effectively counteract the respective upper and lower moments (Mo_{upper arm}, Mo_{lower arm}) can be done in a variety of ways. For example, in some embodiments, springs 130, 144 can be selected to create an opposing force, equal and opposite to that of the upper and lower moments. Specifically, Hooke's law can be applied to determine an approximate spring constant K required of springs 130, 144. Accordingly, in some embodiments, the springs 130, 144 can be appropriately sized to match the respective weights of the user's upper and lower arms (including any expected payloads).

Alternatively or in addition to the selection of springs 130, 144 having a specific constant K, a spring preload can be applied to the springs 130, 144, for example by rotating the upper and lower indexing discs 122, 136 relative to a gravitational field, thereby adjusting a tension of the springs 130, 144 as well as displacement of the first ends 124, 138 of the tension cables 120, 134 relative to pivot points 123, 137. In practice it is been found that changing the spring 130, 144 preload can lead to a non-ideal counterbalance, requiring additional input by the patient. To further tune the upper torso augmentation device 100, one or more movable masses can be added to at least one of the upper and/or lower arm assemblies 102, 106.

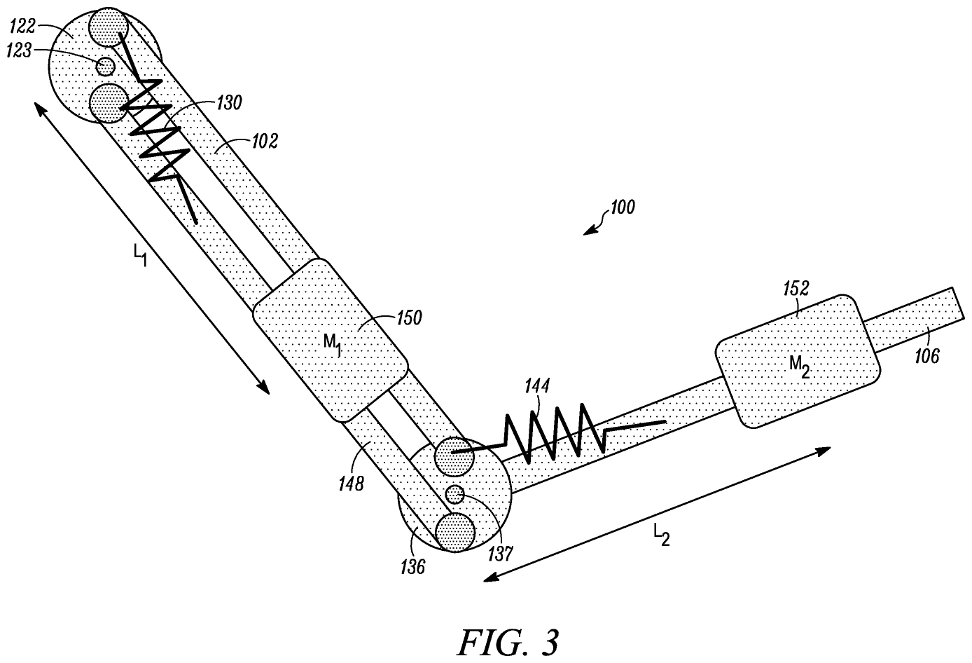

Referring to FIG. 3, an upper torso augmentation device 100 including one or more movable counterbalancing masses 150, 152, is depicted in accordance with an embodiment of the disclosure. Accordingly, rather than adjusting the spring preload, the one or more movable weights 150, 152 can be used to affect a change in the respective upper and lower moments (Mo_{upper arm}, Mo_{lower arm}). That is, rather than tuning the springs 130, 144 to counteract the upper and lower moments, the upper and lower moments can be tuned to a given spring 130, 144.

Accordingly, since respective upper and lower moments can be tuned to spring having a specific constant K and/or pretension, an “ideal” counterbalance can be achieved if a sufficiently sized mass 150, 152 can be moved over a sufficient distance L₁, L₂ (where L₁, L₂ represented distance between the center of gravity of the mass 150, 152 and the pivot points 123, 137. In some embodiments, a spring preload adjustment can be used in combination with one or more movable masses 150, 152 as an aid in achieving an ideal counterbalance. Further, the positions of the masses 150, 152 can also be used to offset the change in moment introduced by a payload in a user's hand, thereby enabling a single spring to counterbalance the user's arm regardless of the payload held in the user hand.

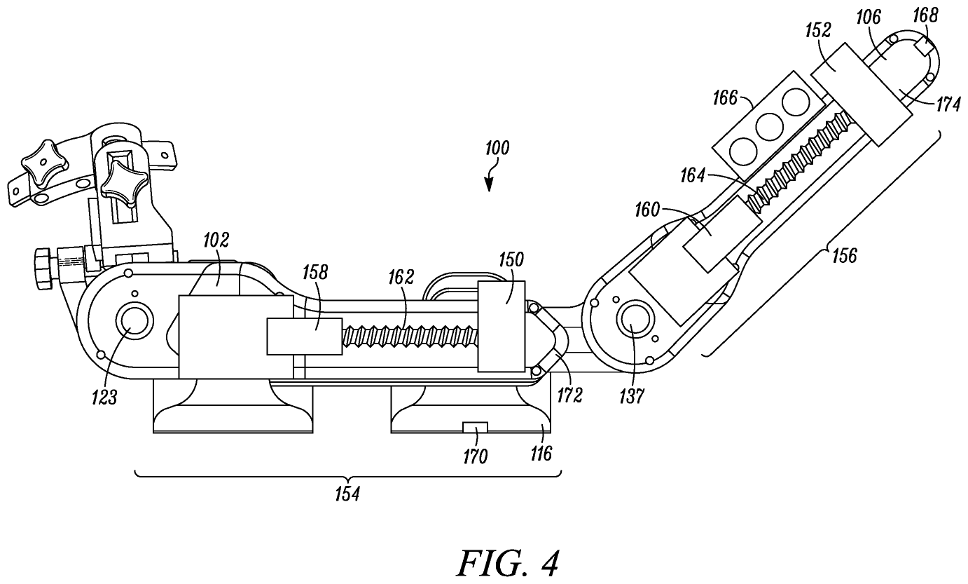

Referring to FIG. 4, an upper torso augmentation device 100 having one or more movable masses to tune the respective upper and lower moments to achieve a more ideal counterbalance for a range of users and payloads held by those users, is depicted in accordance with an embodiment of the disclosure. In some embodiments, the upper torso augmentation device 100 can include an actuation system 154, 156 configured to either manually or automatically position the movable masses 150, 152 to affect a change in the respective upper and lower moments. For example, in one embodiment, can include one or more motors or actuators 158, 160, and one or more linear motion systems 162, 164 (e.g., a lead screw and track, etc.) configured to position the masses 150, 152 at a desired distance from the pivot points 123, 137, thereby adjusting the mechanical moment (e.g., Mo_{upper arm}, Mo_{lower arm}) about the pivot point 123, 137.

In some embodiments, a one-time calibration can be performed to configure the mass 150, 152 (e.g., move the masses 150, 152 a desired distance L₁, L₂ from pivots 123, 137 to achieve desired upper and lower moments) for a user's unique arm. The one-time calibration can include tuning adjusting the distances of the masses 150, 152 in order to create a desired balance in the upper torso augmentation device 100 based on the weight of the user's arm and/or the physical demand/strength profile desired for the patient therapy or treatment. Accordingly, in some embodiments, the movable masses 150, 152 can be utilized to tune the upper torso augmentation device 100 for a specific user.

In other embodiments, the calibration can be performed on a more frequent basis, for example to account for different payloads grasped by the user. For example, in some embodiments, distances L₁, L₂ from can be dynamically controlled by direct user input via a user interface 166 (e.g., via push buttons, sliders, touchscreen, etc.), which can enable a user to adjust the mass 150, 152 positions based on the payload that the user would like to pick up and/or carry. In such an embodiment, the movable masses 150, 152 can be positioned near distal ends 172, 174 of the upper and lower arm assemblies 102, 106. Upon picking up a payload or object, the movable masses 150, 152 can be moved proximately away from the distal ends 172, 174 to ensure that the respective upper and lower moments (Mo_{upper arm}, Mo_{lower arm}) remains substantially unchanged (e.g., the movable masses 150, 152 can shift proximately to reduce the upper and lower moments by approximately the same amount that the picking up the payload increases the upper and lower moments).

In some embodiments, one or more sensors 168 (e.g., wireless sensors) can be utilized to identify known payloads (specifically a known mass of a payload), thereby enabling an electronic actuation system 154, 156 to automatically adjust the position of the movable masses 150, 152 once the payload has been grasped by the user. Accordingly, the system is configured to provide active assistance, by adjusting a position of one or more movable masses 150, 152 dynamically based on the position of the arm and sensed user input to amplify user input, thereby requiring less user strength to overcome friction and misalignment of the device 100 in counteracting the effects of gravity.

In yet another embodiment, adjustment of the mass 150, 152 positions can be based on a sensed patient input force. For example, in one embodiment, one or more load cells 170 (e.g., positioned in at least one of the patient arm cuffs 114, 116, 118) can be configured to monitor forces experienced by the arm of the user over a range of positions. Deviations from an expected force value can be used as an aid in moving the masses 150, 152 to a new location in an effort to achieve a more desirable upper and lower moment.

In some embodiments, the upper torso augmentation device 100 can modify the upper and lower moments to provide active resistance, thereby providing a form of resistance training for a patient by actively opposing the patient input. Users or clinicians can adjust the resistance (or dosage) based on clinical advice. Since the deviation from an ideal counterbalance can be known accurately (known mass, and known distance from pivot or joint), and the amount of movement of the patient's joints can be measured over a course of time, it possible to calculate the amount of work (e.g., force multiplied by displacement) that a patient performs over that period of time. The amount of patient work can be tracked over time, quantifying if the patient strength and endurance is improving or worsening over time, and allowing for controlled experiments to assess the effects of using the device and the dosage of the resistance introduced.

The actuation system 154, 156 can be configured to enable movement of the one or more masses relative to the pivots 123, 137 can have a variety of configurations. For example, in some embodiments, the actuation system 154, 156 can be a sliding mass system (e.g., including masses 150, 152) configured to move along the respective upper and lower arm assemblies 102, 106 (e.g., in tandem) to control the respective upper and lower moments. In other embodiments, the actuation system 154/156 can be a continuous mass transfer system (e.g., in the form of an unevenly weighted chain, transferable fluid, etc.) configured to transfer a mass from an off-arm location to a desired position on the respective upper and lower arm assemblies 102, 106. In embodiments, the actuation system 154, 156 can be adjusted manually via a user or clinician, or the actuation system 154, 156 can be automatically driven (e.g., via one or more motors or actuators 158, 160).

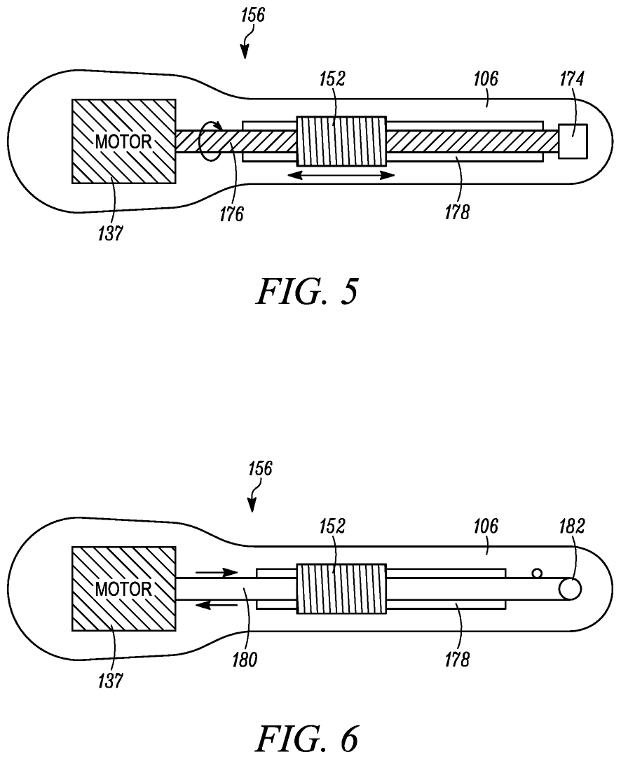

Various examples of sliding mass systems are depicted in FIGS. 5-8. Various embodiments of the present invention will be described in detail with reference to the drawings, wherein like reference numerals represent like parts and assemblies throughout the several views. For example, with reference to FIG. 5, in one embodiment, the actuation system 156 can include a motor 137 configured to rotate a lead screw 176 the mass 152 along a track 178.

With reference to FIG. 6, in another embodiment, the actuation system 156 can be in the form of a pulley and track system configured to drive the mass 152 along a linear path. In this embodiment, the actuation system 156 can include a cable 180 operably coupled to the mass 152 and driven with one or more pulley wheels 182 operably coupled to the motor 137. In some embodiments, the cable can be constructed of a polymer, fiber or metal material. In other embodiments, the cable 180 can be replaced with a flexible toothed belt driven with a toothed pulley wheel operably coupled to the motor 137.

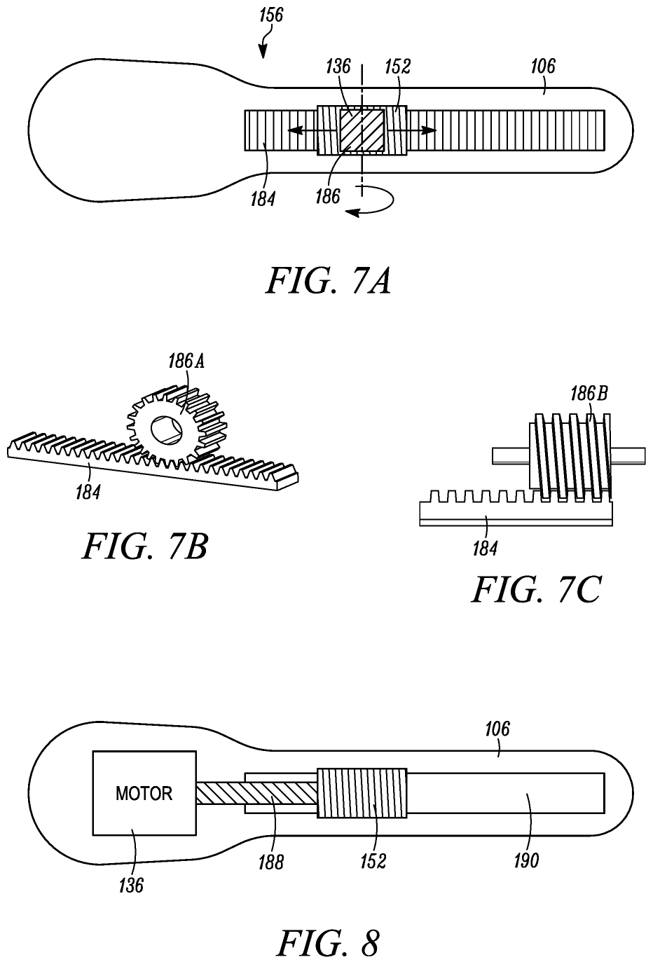

With reference to FIG. 7A, in another embodiment, the actuation system 156 can be a rack and pinion system or other similar geared assembly configured to drive the mass 152 along a linear path. With additional reference to FIGS. 7B-C, in this embodiment, the actuation system 156 can include a rack 184 and pinion 186A (as depicted in FIG. 7B) or worm gear 186B (as depicted in FIG. 7C). The motor 137 can be configured to rotate the pinion or worm gear 186 in order to affect a linear motion of the mass 152 relative to the arm assembly 106. As a variant of this type of system, in another embodiment, the actuation system 156 can be a friction-based drive system, for example including one or more rubber wheels driven configured to rotatably engage with a track as the mass is driven down the track, wherein frictional forces between the rubber wheels and track inhibit the rubber wheels from sliding relative to the track.

With reference to FIG. 8, in another embodiment, the actuation system 156 can include a resilient push pull linkage 188, for example in the form of a coil or tape which can be selectively wound and unwound around a spool or drum. For example, in one embodiment, the resilient push pull linkage 188 can be in the form of a flexible wire spooled around a drum that can be rotated by a motor 136 to extend or retract the push pull linkage 188 along a confined channel 190. The movable mass 152 can be operably coupled to an end of the push pull linkage 188, thereby enabling the mass 152 to be linearly translated along the arm assembly 106.

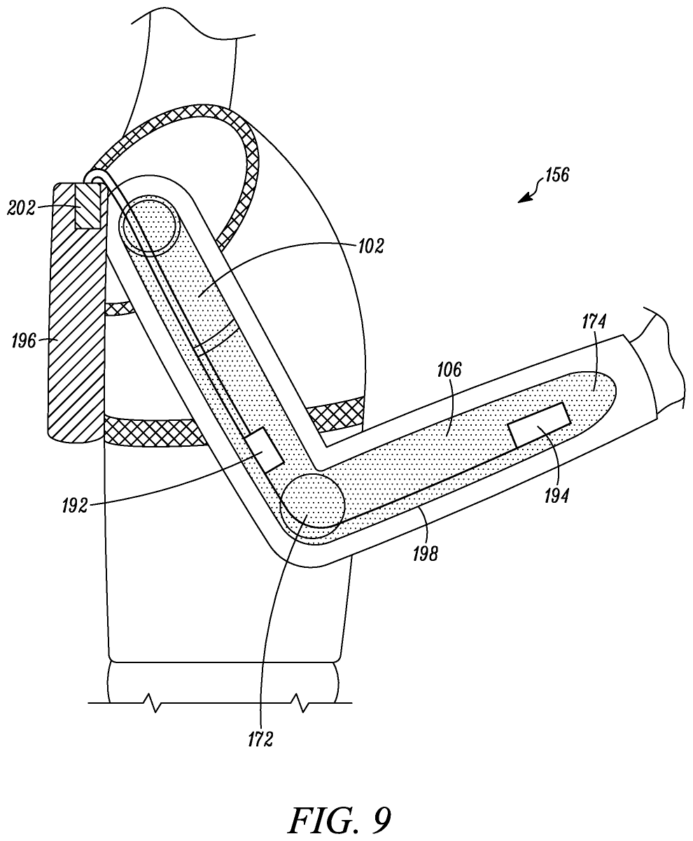

Various examples of continuous mass transfer systems are depicted in FIG. 9-12. Various embodiments of the present invention will be described in detail with reference to the drawings, wherein like reference numerals represent like parts and assemblies throughout the several views. For example, with reference to FIG. 9, in one embodiment, a liquid or fluid can be transferred between one or more arm reservoirs 192, 194 (e.g., positioned proximity to respective distal ends 172, 174 of the upper and lower arm assemblies 102, 106) and a storage reservoir 196 (e.g., an off-arm storage reservoir) via one or more fluid transfer lines 198 and a fluid pump 202. As fluid is transferred from the storage reservoir 196 to the one or more arm reservoirs 192, 194, we can be shifted to the upper and lower arm assemblies 102, 106, thereby modifying the upper and lower moments. In some embodiments, the actuation system 156 can employ a single adjustable weight reservoir 194 located in proximity to a user's wrist or forearm, large enough to alter the moment of both the upper and lower arm assemblies 102, 106. In a variant of this embodiment, the fluid can be replaced by a solid media (e.g., a plurality of spheres), which can be moved from one or more counterbalancing reservoirs 196 distally along the upper and lower arm assemblies 102, 106 along the transfer line 198 to affect a change in the upper and lower moments. Such embodiments can include a feed screw with an electric motor (e.g., positioned at both ends of the transfer line 198).

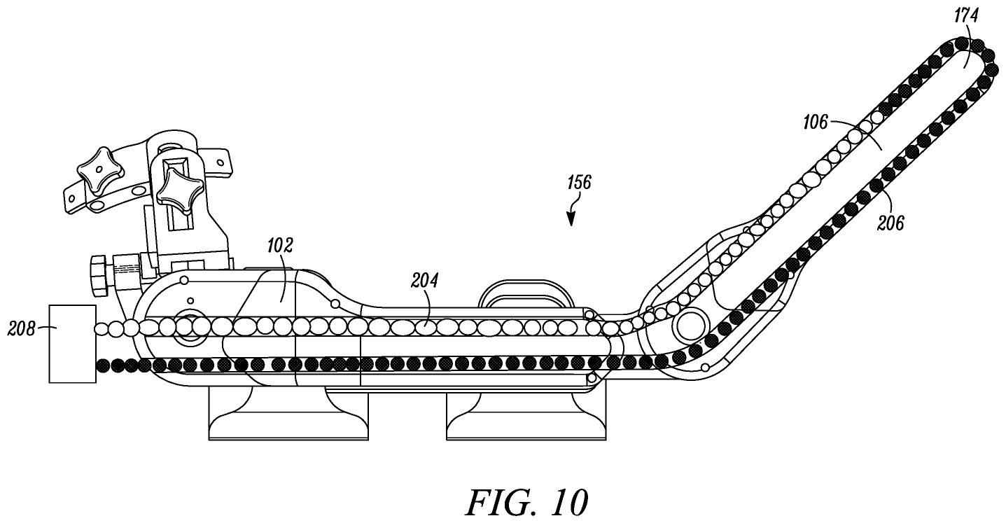

With reference to FIG. 10, in another embodiment, the actuation system 156 can include a continuous chain of solid media 204 having multiple or variable densities, including at least a first density portion and a second density portion, wherein the first density portion has a higher density than the second density portion. The media 204 can be arranged such that the first density portion is grouped together collectively as a movable mass 206. Accordingly, the upper and lower moments of the upper and lower arm assemblies 102, 106 can be adjusted by movement of the continuous chain of solid media 204, as the movable mass 206 moves relative to the distal end 174 of the upper torso augmentation device 100. Such an embodiment can include one or more feed screw with electric motor 208 to move the solid media 204 along a channel within the upper and lower arm assemblies 102, 106.

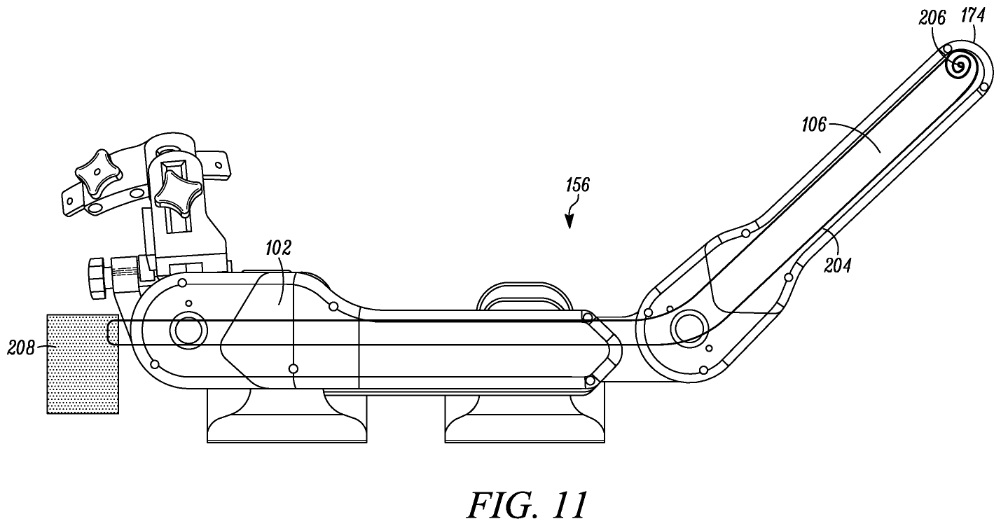

With reference to FIG. 11, in another embodiment, the actuation system 156 can include a continuous chain of solid media 204 configured to be coiled in proximity to a distal end 174 of the upper torso augmentation device 100. Like the previous embodiment, the solid media 204 can have multiple densities, wherein a first density portion having a higher density than a second density portion is grouped together collectively as a movable mass 206. To affect a change in the upper and lower moments, the movable mass 206 can be moved relative to the distal end 174 of the upper torso augmentation device 100. Such an embodiment can include an electric motor 208 with a drive pulley and a channel or tubing defined within the upper and lower arm assemblies 102, 106 to enable the solid media 204 to pass therethrough. In some embodiments, the solid media 204 can act as both the movable mass 206 and a drive system for the upper torso augmentation device 100.

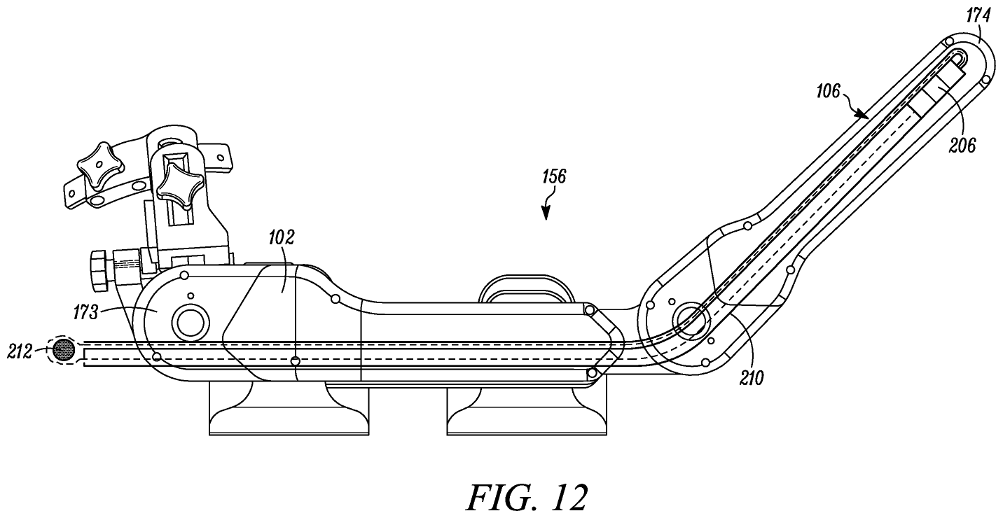

With reference to FIG. 12, in yet another embodiment, the actuation system 156 can include a rigid movable mass 206 configured to be transferred between a proximal end 173 and distal end 174 of the upper torso augmentation device 100. In embodiments, the movable mass 206 can be transferred via a rope or cable 210 configured to circulate a path from a remote location in proximity to the proximal and 173 (e.g., an off-arm location) to a portion of the upper or lower arm assembly 102, 106. Control and adjustment of the upper and lower moments can be affected by movement of the movable mass toward the distal end 174 of the upper torso augmentation device 100. Such embodiments can include an electric motor and drive pulley 212 with the movable mass 206 and cable travelling within a channel or tubing defined by the upper and lower arm assemblies 102, 106. Alternative mechanisms for moving the weights or masses without the use of any motors, pumps, or other “powered” drivers are also contemplated. For example, in one embodiment, the masses could be moved on a slide using cables to pull the movable masses along the upper and lower arm assemblies 102, 106 without the use of actively powered mechanisms (e.g., the masses can be moved manually).

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. An upper torso augmentation device in which a moment of an arm assembly is tunable by the movement of one or more movable masses, the upper torso augmentation device comprising:
an upper arm assembly pivotably coupled to a shoulder assembly, the upper arm assembly including an assisted force mechanism configured to aid in counteracting an effect of gravity upon the upper arm assembly and any payload carried thereby, wherein the assisted force mechanism comprises one or more movable masses configured to move relative to a distal end of the upper arm assembly, thereby affecting a change in a moment of the upper arm assembly.

2. The upper torso augmentation device of claim 1, wherein the assisted force mechanism comprises at least one spring.

3. The upper torso augmentation device of claim 1, wherein a tension in the at least one spring is adjustable via a pre-tensioning mechanism.

4. The upper torso augmentation device of claim 1, further comprising a lower arm assembly pivotably coupled to the upper arm assembly, the lower arm assembly including a second assisted force mechanism configured to aid in counteracting an effect of gravity upon the lower arm assembly and any payload carried thereby, wherein the second assisted force mechanism comprises one or more lower arm movable masses configured to move relative to a distal end of the lower arm assembly, thereby affecting a change in moment of the lower arm assembly.

5. The upper torso augmentation device of claim 1, wherein the one or more movable masses are moved via at least one of a manual or automated actuation system.

6. The upper torso augmentation device of claim 1, wherein the assisted force mechanism is controllable via a user interface.

7. The upper torso augmentation device of claim 1, wherein the assisted force mechanism further comprises one or more sensor configured to identify known payloads for automatic movement of the one or more movable masses.

8. The upper torso augmentation device of claim 1, wherein the assisted force mechanism includes one or more load cells configured to monitor forces experienced in an arm of a user, wherein a deviation from an expected force value triggers automatic movement of the one or more movable masses.

9. The upper torso augmentation device of claim 1, wherein the assisted force mechanism is configured to provide active resistance as a form of resistance training.

10. The upper torso augmentation device of claim 1, wherein the assisted force mechanism is configured to calculate an amount of work performed by a user over a defined period of time.

11. An upper torso augmentation device, comprising:
at least one arm assembly including an assisted force mechanism configured to counteract an effect of gravity upon an arm of a user through a desired range of motion, the assisted force mechanism comprising one or more movable masses configured to move relative to a distal end of the at least one arm assembly, thereby affecting a change in moment of the at least one arm assembly.

12. The upper torso augmentation device of claim 11, wherein the assisted force mechanism includes an actuation system comprising a rotatable lead screw to shift the one or more movable masses along a track.

13. The upper torso augmentation device of claim 11, wherein the assisted force mechanism includes an actuation system comprising a pulley wheel system configured to drive a cable upon which the one or more movable masses is attached in order to affect movement in the one or more movable masses along a length of the at least one arm assembly.

14. The upper torso augmentation device of claim 11, wherein the assisted force mechanism includes an actuation system comprising a rack and pinion system configured to affect movement in the one or more movable masses along a length of the at least one arm assembly.

15. The upper torso augmentation device of claim 11, wherein the assisted force mechanism includes an actuation system comprising a resilient push pull linkage configured to affect movement in the one or more movable masses along the length of the at least one arm assembly.
